(19) United States
(12) Patent Application Publication
(10) Pub. No.: US 2025/0332341 A1
(43) Pub. Date: Oct. 30, 2025

(54) FLUID MANAGEMENT SYSTEM WITH FLOATING PUMP

(71) Applicant: BOSTON SCIENTIFIC SCIMED, INC., Maple Grove, MN (US)

(72) Inventors: Linda Maher, Boston, MA (US); Peter Joyce, Tipperary (IE); Joseph Patrick Hickey, Tullow (IE); Thomas O'Brien, Cork (IE); John Prior, Tipperary (IE); Edwin Varghese Chiriyankandath, Tipperary (IE)

(73) Assignee: BOSTON SCIENTIFIC SCIMED, INC., Maple Grove, MN (US)

(21) Appl. No.: 19/193,331

(22) Filed: Apr. 29, 2025

Related U.S. Application Data
(60) Provisional application No. 63/640,601, filed on Apr. 30, 2024.

(51) Int. Cl. A61M 5/142; A61B 1/015; A61M 5/14; A61M 5/168

(52) U.S. Cl. CPC A61M 5/14232; A61M 5/1413; A61M 5/16813; A61B 1/015; A61M 2205/3331; A61M 2205/36; A61M 2205/505

(57) ABSTRACT

A fluid management system including a console including a roller pump and a controller for operating the roller pump. The console includes a housing and a door movable between a closed position and an open position. The roller pump is rotatable about a rotational axis during operation of the roller pump, and the rotational axis is configured to float along an arcuate pathway relative to the housing during operation of the roller pump. The roller pump is configured to pivot relative to the housing about a pivot axis during operation of the roller pump. The pivot axis is spaced apart from the rotational axis of the roller pump.

### CROSS-REFERENCE TO RELATED APPLICATIONS

The application claims the benefit of U.S. Provisional Patent Application Ser. No. 63/640,601, filed on Apr. 30, 2024, the disclosure of which is incorporated herein by reference.

### TECHNICAL FIELD

The disclosure is directed to a fluid management system. More particularly, the disclosure is directed to a fluid management system pump console and associated a disposable fluid cassette configured to engage a roller pump of the fluid management system pump console.

### BACKGROUND

Flexible ureteroscopy (fURS), gynecology, and other endoscopic procedures require the circulation of fluid for several reasons. Fluid management systems may be used to deliver fluid to an anatomical cite from a reservoir at a desired pressure and/or flow rate via a peristaltic or roller pump. Fluid management systems may adjust the flow rate and/or pressure at which fluid is delivered from the reservoir based on data collected from a procedural device, such as, but not limited to, pressure readings sensed and/or obtained by the fluid management system. The fluid management system may utilize a disposable fluid tubing set installed with a pump console to provide the fluid to the patient. There is an ongoing need to provide alternative configurations of the components of fluid management systems, to facilitate the use thereof.

### BRIEF SUMMARY

This disclosure provides design, material, manufacturing method, and use alternatives for components of a fluid management system.

A first example is a fluid management system. The fluid management system includes a console including a roller pump and a controller for operating the roller pump. The console includes a housing and a door movable between a closed position and an open position. The roller pump is rotatable about a rotational axis during operation of the roller pump. The rotational axis is configured to float along an arcuate pathway relative to the housing during operation of the roller pump.

Alternatively or additionally to any of the examples above, in another example, the fluid management system includes a fluid cassette insertable into a receptacle of the housing of the console. The fluid cassette includes a length of flexible pump tubing configured to engage the roller pump.

Alternatively or additionally to any of the examples above, in another example, the flexible pump tubing extends in an arcuate pathway in which the pump tubing extends outward beyond a front face of a housing of the fluid cassette when engaged with the roller pump.

Alternatively or additionally to any of the examples above, in another example, the door includes an occlusion bed mounted on an interior surface of the door, wherein the occlusion bed is configured to engage the length of flexible pump tubing of the fluid cassette when the door is closed to compress the length of flexible pump tubing between the occlusion bed and the roller pump.

Alternatively or additionally to any of the examples above, in another example, the roller pump is configured to apply a force against the length of flexible pump tubing in the range of 47.5 Newtons (N) to 74.5 N.

Alternatively or additionally to any of the examples above, in another example, the roller pump is rotatably mounted on a pump mount, wherein the pump mount is pivotably mounted to the housing.

Alternatively or additionally to any of the examples above, in another example, the pump mount includes a pivot point, a first arm extending from the pivot point to the roller pump, and a second arm extending from the pivot point away from the roller pump.

Alternatively or additionally to any of the examples above, in another example, the fluid management system includes a spring exerting a force against the second arm of the pump mount.

Alternatively or additionally to any of the examples above, in another example, the spring is replaceable via a threaded plug of the housing of the console.

Alternatively or additionally to any of the examples above, in another example, the fluid management system includes a motor driving the roller pump, wherein the motor moves along the arcuate pathway with the roller pump during operation of the roller pump.

Alternatively or additionally to any of the examples above, in another example, the fluid management system includes a fluid-tight containment housing surrounding the roller pump, wherein the fluid-tight containment housing is configured to isolate the controller and associated electronic circuitry of the console from fluid.

Another example is a fluid management system. The fluid management system includes a console including a roller pump and a controller for operating the roller pump. The console includes a housing and a door movable between a closed position and an open position. The roller pump is rotatable about a rotational axis during operation of the roller pump. The roller pump is configured to pivot relative to the housing about a pivot axis during operation of the roller pump, the pivot axis being spaced apart from the rotational axis.

Alternatively or additionally to any of the examples above, in another example, the pivot axis is parallel to the rotational axis.

Alternatively or additionally to any of the examples above, in another example, the fluid management system includes a fluid cassette insertable into a receptacle of the housing of the console. The fluid cassette includes a length of flexible pump tubing configured to engage the roller pump.

Alternatively or additionally to any of the examples above, in another example, the flexible pump tubing extends in an arcuate pathway along roller pump.

Alternatively or additionally to any of the examples above, in another example, the door includes an occlusion bed mounted on an interior surface of the door, wherein the occlusion bed is configured to engage the length of flexible pump tubing of the fluid cassette when the door is closed to compress the length of flexible pump tubing between the occlusion bed and the roller pump.

Alternatively or additionally to any of the examples above, in another example, the roller pump is configured to apply a force against the length of flexible pump tubing in the range of 47.5 Newtons (N) to 74.5 N.

Alternatively or additionally to any of the examples above, in another example, the fluid management system includes a fluid-tight containment housing surrounding the roller pump, wherein the fluid-tight containment housing is configured to isolate the controller and associated electronic circuitry of the console from fluid.

Another example is method of operating a fluid management system. The method includes positioning a housing of a fluid cassette of a disposable fluid tubing set into a receptacle of a housing of a console of the fluid management system with a length of flexible pump tubing of the fluid cassette engaged against a roller pump of the console. The method further includes closing a door of the console such that an occlusion bed on an interior wall of the door compresses the length of flexible pump tubing between the occlusion bed and the roller pump. A rotational axis of the roller pump is configured to float along an arcuate pathway relative to the housing during operation of the roller pump.

Alternatively or additionally to any of the examples above, in another example, the roller pump is configured to apply a force against the length of flexible pump tubing in the range of 47.5 Newtons (N) to 74.5 N.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set;

FIG. 3 is perspective view of the rear side of the fluid cassette of the disposable fluid tubing set of FIG. 2;

FIG. 4 is a perspective view of the fluid cassette and console during insertion/ejection of the fluid cassette;

FIG. 5 is a perspective view of the receptacle of the console including a roller pump configured to engage tubing of the fluid cassette;

FIG. 6 is perspective view of the roller pump, and associated motor and mounting assembly;

FIG. 7 is a top view of the roller pump, and associated motor and mounting assembly;

FIG. 8 is side view of the interaction of the roller pump with tubing of the fluid cassette; and

FIG. 9 is a perspective view of a portion of the roller pump and associated mounting assembly, including a fluid-tight containment housing.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may control the flow of fluid into the body cavity and/or regulate body cavity pressure and/or the flow rate of fluid flow to the body cavity using an inflow and/or outflow pump of the fluid management system. The inflow pump may deliver fluid through inflow tubing of a fluid tubing set to the patient and/or the outflow pump may remove fluid through outflow tubing of a fluid tubing set from the patient. The fluid management system may include one or more sensors providing signals to the controller of the fluid management system to control the fluid flow.

In some instances in which the fluid management system is used in conjunction with an endoscope device such as, but not limited to, a LithoVue™ Elite endoscope, the fluid management system may control the fluid flow using pressure and/or temperature data from the endoscope or other endoscopic device. Direct regulation of the intracavity pressure during a medical procedure using a pressure sensor on the endoscope may allow the fluid management system to safely control the fluid pressure with the body cavity.

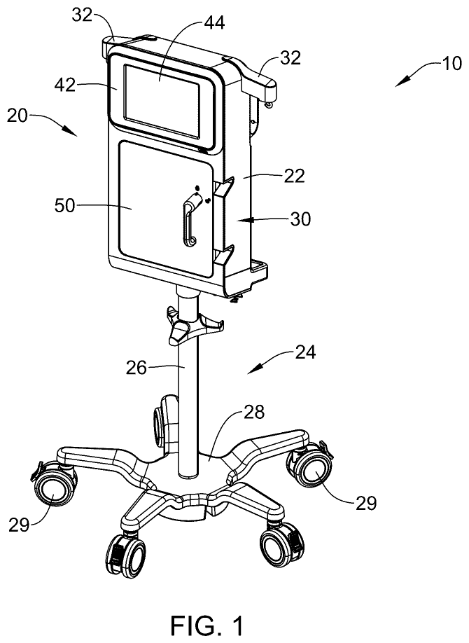

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the Litho Vue™ Elite endoscope, or other endoscope. In some instances, the endoscope may include a temperature sensor to provide intracavity temperature feedback to the fluid management system 10, a pressure sensor to provide intracavity pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28. The base 28 may include a plurality of wheels 29 (e.g., caster wheels), allowing the cart 24 to be wheeled around to a desired location. In other instances, the console 20 may be provided with another form of cart, configured to be positioned on a flat surface, mounted to a wall, etc.

The fluid management system 10 may also include one or more user interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The touch screen interface 42 allows the user to input/adjust various functions of the fluid management system 10 such as, for example flow rate, pressure, and/or temperature. The user may also configure parameters and alarms (such as, but not limited to, a max pressure alarm), information to be displayed, and the procedure mode. The touch screen interface 42 allows the user to add, change, and/or discontinue the use of various modular systems within the fluid management system 10. The touch screen interface 42 may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flow rate and/or fluid pressure display. In some embodiments, operating parameters may be adjusted by touching a corresponding portion of the touch screen interface 42. The touch screen interface 42 may also display visual alerts and/or audio alarms if parameters (e.g., flow rate, temperature, etc.) are above or below predetermined thresholds and/or ranges. In some embodiments, the fluid management system 10 may also include further user interface components such as an optional foot pedal, a fluid warmer user interface, a fluid control interface, or other device to manually control various modular systems. For example, an optional foot pedal may be used to manually control flow rate. Some illustrative display screens 44 and other user interface components are described in described in commonly assigned U.S.

Patent Application Publication No. 2018/0361055, titled AUTOMATED FLUID MANAGEMENT SYSTEM, the entire disclosure of which is hereby incorporated by reference.

The touch screen interface 42 may be operatively connected to or a part of the controller 30. The controller 30 may be a CPU, including a computer, tablet computer, or other processing device. The controller 30 may be operatively connected to one or more system components such as, for example, an inflow pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flow rate or the fluid pressure of fluid provided by the fluid management system 10 at any given time may be displayed on the display screen 44 to allow the operating room (OR) visibility for any changes. If the OR personnel notice a change in fluid flow rate or fluid pressure that is either too high or too low, the user may manually adjust the fluid flow rate or the fluid pressure back to a preferred level. The fluid management system 10 may also monitor and automatically adjust the fluid flow rate or the fluid pressure based on previously set parameters, as discussed herein.

An illustrative fluid management unit may include one or more fluid container supports, such as fluid supply source hanger(s) 32, each of which may support a fluid supply source (e.g., fluid bag). In some embodiments, placement and/or weight of the fluid supply source(s) hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more collection containers (not shown), for collecting waste fluid during a medical procedure. The collection containers (e.g., canisters) may be in fluid communication with a vacuum pump to provide suction for drawing fluid into the collection containers. The vacuum pump may be operatively and/or electronically connected to the controller 30. In some embodiments, the vacuum pump may be disposed within the fluid management system 10. Other configurations are also contemplated. In some embodiments, the collection container(s) may be operatively coupled to a collection load cell to detect placement and/or weight of fluid in the collection container(s) to contribute to a fluid deficit calculation.

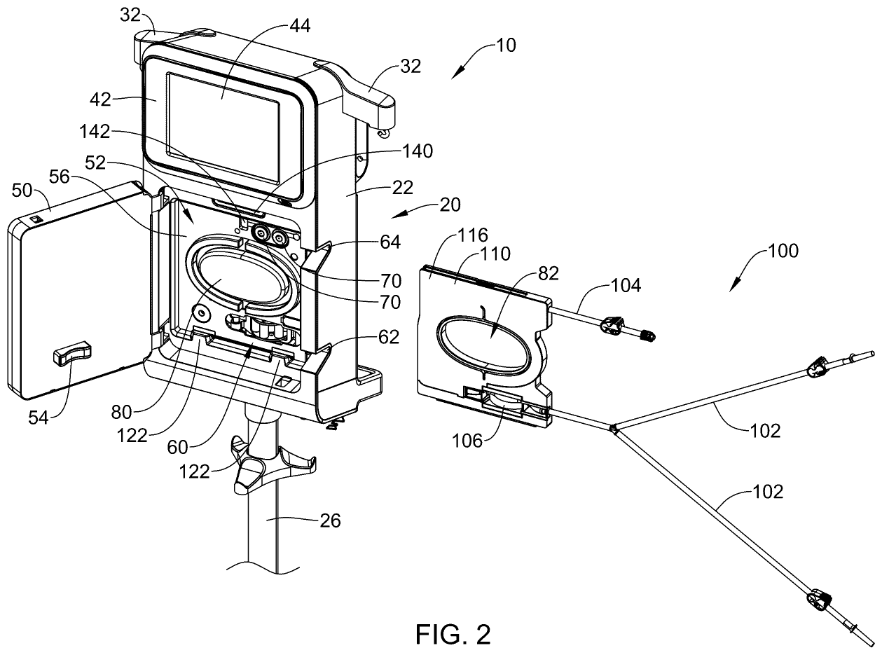

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The receptacle 52 may include a rear wall 56. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. A portion of the inflow pump 60 may extend through an opening in the rear wall 56 of the receptacle to engage the length of flexible pump tubing 106, while a remaining portion of the inflow pump 60, as well as the motor powering the inflow pump 60 may be located on the rear side of the rear wall 56. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flow rate or a target fluid pressure. As noted herein, the inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target fluid flow rate or target fluid pressure based on a set of system operating parameters. In some embodiments, the controller 30 may be configured to control the inflow pump 60 to maintain a desired fluid pressure at the treatment site or a desired flow rate based on a set of system operating parameters.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flow rate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flow rate, fluid pressure, and/or other parameters. Once the user has selected the manual mode on, for example, the touch screen interface 42, the user may the adjust fluid flow rate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the fluid inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike, or other connector for connecting to the fluid supply source(s). The single use tubing set 100 may also include outflow tubing 104 providing a fluid outflow from the interior of the cassette 110 to a medical device connected thereto. The single use tubing set 100, including the fluid cassette 110, the fluid inflow tubing 102, and the fluid outflow tubing 104, may be disposable and provided sterile and ready to use.

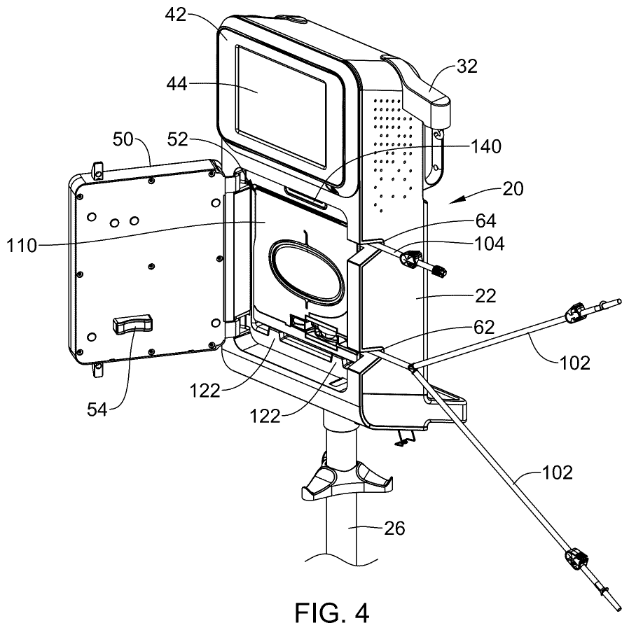

When the fluid cassette 110 is installed in the receptacle 52 and the door 50 is closed, the inflow tubing 102 may pass through a channel 62 extending through a wall of the housing 22 of the console 20 to an exterior of the console 20 (see FIG. 4). Likewise, when the fluid cassette 110 is installed in the receptacle 52 and the door 50 is closed, the outflow tubing 104 may pass through a channel 64 extending through a wall of the housing 22 of the console to an exterior of the console 20 (see FIG. 4). The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flow rates required in the specific application of the fluid management system 10. Some illustrative fluid warming systems are described in described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

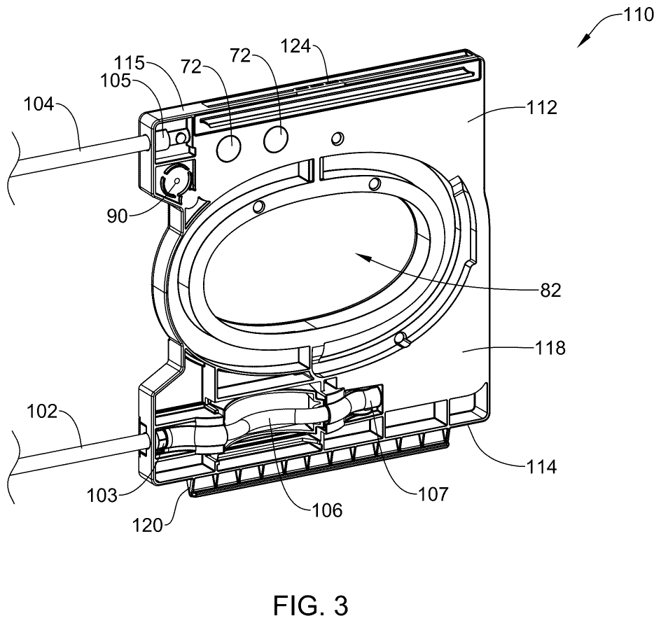

The console 20 may further include one or more additional sensors, such as a pressure sensor and/or a bubble sensor. For instance, the console 20 may include a pressure sensor 70, illustrated as a pair of pressure sensors, configured to monitor a system pressure of fluid exiting the cassette 110 and flowing through the outflow tubing 104 to a surgical site. The fluid cassette 110 may include a corresponding pressure sensor interface 72, such as a flexible membrane, (shown in FIG. 3) that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44.

Additional features of the cassette 110 of the fluid tubing set 100 are illustrated in FIG. 3. The fluid cassette 110 may include a housing 112 defining a fluid pathway through an interior of the housing 112. The fluid cassette 110 may include a front face 116 and a rear face 118 opposite the front face 116. The front face 116 is configured to face the door 50 of the console 20 when loaded in the receptacle 52, and the rear face 118 is configured to face a rear wall 56 of the receptacle 52 of the console 20 when loaded in the receptacle 52. The fluid cassette 110 may also include an upper edge 115 and a lower edge 114 opposite the upper edge 115. The housing 112 of the fluid cassette 110 may also include an opening 82, such as an oval opening, extending through the housing 112 from the front face 116 to the rear face 118. The opening 82 may extend a majority of the length of the housing 112 (i.e., a majority of the distance between the lateral edges of the housing 112) and/or a majority of the height of the housing 112 (i.e., a majority of the distance between the upper edge and the lower edge of the housing 112), in some instances. The opening 82 may be configured to receive an elevated portion of the rear wall 56 of the receptacle 52, shown in FIG. 1 as the fluid warming system 80. The elevated portion of the rear wall 56 of the receptacle 52 may be an oval shape sized to fit through the oval shaped opening 82 of the housing 112 of the fluid cassette 110 when the fluid cassette 110 is in its loaded position in the receptacle 52. In embodiments, in which the console 20 lacks a fluid warming system, the elevated portion of the rear wall 56 of the receptacle 52 may still be present. Insertion of the elevated portion of the rear wall 56 of the receptacle 52 through the opening 82 of the fluid cassette 110 may facilitate proper alignment of the fluid cassette 110 in the receptacle 52, for example.

In some embodiments, the fluid cassette 110 may include a fluid inlet port 103 and a fluid outlet port 105 located at a lateral side of the fluid cassette 110. The fluid inlet port 103 may be coupled to the inflow tubing 102 and the fluid outlet port 105 may be coupled to the outflow tubing 104. The cassette 110 may define an internal fluid pathway through an interior of the cassette housing 112 of the cassette 110 between the fluid inlet port 103 and the fluid outlet port 105. In embodiments of the fluid management system 10 including fluid warming capabilities, the internal fluid pathway may include the susceptor. The length of flexible pump tubing 106 of the cassette 110, configured to engage and be compressed by the rollers of the inflow pump 60, may extend from the fluid inlet port 103 to a connection 107 of the cassette 110 leading to the fluid pathway defined through the interior of the cassette 110. The flexible pump tubing 106 may be a discrete length of tubing separate from the inflow tubing 102 and the outflow tubing 104. The flexible pump tubing 106 may have a fixed or controlled length to ensure a constant and/or consistent flow rate is achieved through the fluid cassette 110, including the outflow tubing 104. In some instances, the length of the flexible pump tubing 106 may be 160 millimeters (mm)+4 mm, 162 mm+2 mm, or 162 mm+1 mm, for example. In some instances, the flexible pump tubing 106 may extend through an arcuate pathway between the fluid inlet port 103 to the connection 107, such that the flexible pump tubing 106 follows the rotational path of the rollers of the inflow pump 60. The inlet port 103, the outlet port 105, and/or the connection 107 may be formed as a portion of the cassette housing 112, or formed separately and connected thereto.

The fluid cassette 110 may also include an air vent valve 90 configured to release air from the interior of the fluid cassette 110 to atmosphere. For example, the fluid cassette 110 may include an air vent including a hydrophobic membrane, allowing air, including bubbles entrained in the fluid, to pass through the hydrophobic membrane while preventing fluid within the fluid cassette 110 to pass therethrough. The air may then be vented to atmosphere through the air vent valve 90.

The fluid cassette 110 may also include one or more retention features configured to interact with the console 20 to retain the fluid cassette 110 in the receptacle 52 of the console 20. For example, the fluid cassette 110 may include a retention tab 120 extending from a lower edge of the housing 112 of the fluid cassette 110 and/or a retention tab 124 extending from an upper edge 115 of the housing 112 of the fluid cassette 110, configured to engage mating retention features of the console 20, as will be described in more detail below.

The retention tab 120 may interact with a retention tab 122 (See FIG. 2) of the console 20 to hold the fluid cassette 110 in an ejected position when the door 50 is opened, without the cassette 110 falling out of the receptacle 52 of the console 20. For example, when the door 50 is opened, the cassette 110 may tilt outward from the receptacle 52 at an acute angle to vertical, without the cassette 110 falling completely out of the receptacle 52. In other words, when the door 50 is opened, the cassette 110 may tilt outward from a fully loaded position in which the cassette 110 is vertically loaded into the receptacle 52 of the console 20 to an ejected position, in which the upper edge of the cassette 110 has moved laterally out from the receptacle 52 while the lower edge of the cassette 110 remains in the receptacle 52. Interaction of the retention tab 120 of the fluid cassette 110 with the retention tab 122 of the console 20 may retain the cassette 110 at an acute angle to vertical in the ejected position when the door 50 is opened. In some instances, the acute angle may be in the range of about 20° to about 50°, in the range of about 20° to about 40°, in the range of about 25° to about 35°, or about 30°, for example. Thus, the interaction of the retention tab 120 with the retention tab 122 prevents the cassette 110 from falling completely out of the receptacle 52 due to gravity when the door 50 is opened. In other words, the interaction of the retention tab 120 with the retention tab 122 supports the cassette 110 in the ejected position at the acute angle to vertical without allowing the cassette 110 to fall completely out of the receptacle 52 due to gravity. Furthermore, the interaction of the retention tab 120 with the retention tab 122 allows the cassette 110 to be installed in the receptacle 52 in the ejected position and presented for use, prior to closing the door 50.

As the door 50 is closed or prior to closing the door 50, the housing 112 of the fluid cassette 110 will tilt to a vertical position (a loaded position) in the receptacle 52 with the housing 112 of the fluid cassette 110 lying in a vertical plane, as shown in FIG. 4. The door 50 may be closed with the door 50 facing the front face 116 of the fluid cassette 110 and the rear face 118 of the fluid cassette 110 may be facing the rear wall 56 of the receptacle 52. Furthermore, when opening the door 50 subsequent to use of the fluid cassette 110, the fluid cassette 110 may tilt out of the receptacle 52 without falling completely out of the receptacle 52 due to gravity. Thus, the interaction of the retention tab 120 with the retention tab 122 of the console 20 helps support the cassette 110 in the ejected position at the acute angle to vertical without allowing the cassette 110 to fall completely out of the receptacle 52 due to gravity.

The console 20 may also include a retention mechanism configured to releasably engage the fluid cassette 110 to hold the fluid cassette 110 in the receptacle 52 in a loaded, vertical position, in which the fluid cassette 110 is ready for use. For example, the retention mechanism may be configured to releasably engage an upper portion of the housing 112 of the fluid cassette 110. In a locked position, the retention mechanism may prevent the fluid cassette 110 from tilting outward, thus holding the fluid cassette 110 in the loaded, vertical position in the receptacle 52. For instance, the console 20 may include a latch 142 (see FIG. 2) configured to engage the retention tab 124 on the upper edge 115 of the housing 112 of the fluid cassette 110 when in the loaded, vertical position in the receptacle 52. The retention mechanism may also include an actuator, such as the button 140 (see FIGS. 2 and 4), that may be actuated to release the latch 142 from the retention tab 124. Depressing the button 140 may pivot the latch 142 about a pivot axis to disengage the latch 142 from the retention tab 124. The retention mechanism may include a spring, such as a torsion spring biasing the latch 142 to a locked position in which the latch 142 engages the retention tab 124. Depressing the button 140 overcomes the biasing force of the torsion spring to move the latch 142 from the locked position to an unlocked position in which the latch 142 is disengaged from the retention tab 124.

The console 20 may include one or more sensors configured to confirm whether the fluid cassette 110 has been properly positioned in the receptacle 52 and the door 50 closed prior to permitting operation of the inflow pump 60. For example, the console 20 may include one or more proximity sensors, contact sensors, magnetic sensors, or the like to determine whether the fluid cassette 110 is properly positioned in the receptacle 52 prior to permitting the controller 30 to start the inflow pump 60. Once properly positioned, and the door 50 is closed, the controller 30 may initiate operation of the inflow pump 60 to begin supplying fluid to a treatment site during a medical procedure.

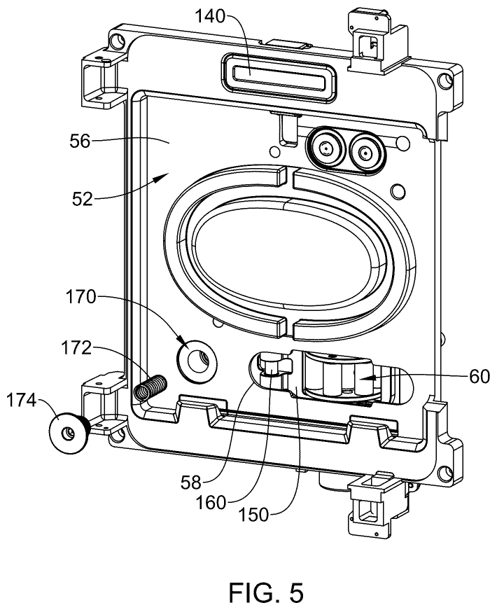

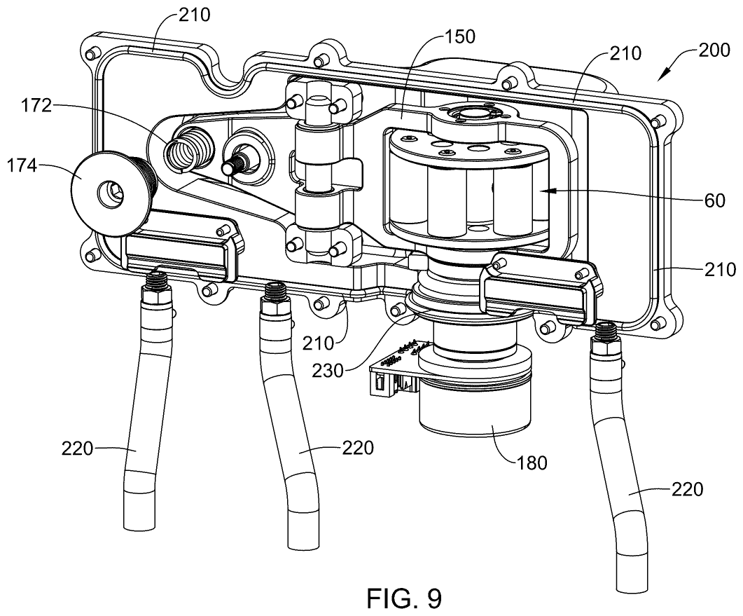

FIG. 5 is a perspective view of the rear wall 56 of the receptacle 52 of the console 20 including a portion of the inflow pump 60 (e.g., roller pump) extending through an opening 58 of the rear wall 56 to permit the inflow pump 60 to engage the flexible pump tubing 106 of the fluid cassette 110. It is noted that the remainder of the inflow pump 60 may be positioned behind the rear wall 56, and within a fluid-tight containment housing 200, as discussed herein in association with FIG. 9. The inflow pump 60 may be rotatably mounted to a pump mount 150. The pump mount 150 may be pivotably mounted to the housing of the console 20 via a pivot pin 160, or other structure permitting the pump mount 150, and thus the inflow pump 60, to float (e.g., pivot about a pivot axis) relative to the housing 22 of the console 20, and the fluid cassette 110 loaded therein, during operation of the inflow pump 60.

The rear wall 56 of the receptacle 52 may include an access opening 170 providing access to install, repair, inspect, adjust and/or replace a spring 172. A plug 174, such as a threaded plug including a threaded stem 175 configured to threadably engage a threaded portion of the access opening 170, may be provided to access the spring 172 disposed in the access opening 170. The plug 174 may be removed from the access opening 170 to access the spring 172 to inspect and/or replace the spring 172, for instance. In some instances, the spring 172 may have a service life (e.g., 1 year, for example), and thus should be replaced with a new spring 172 at the conclusion of the service life of the used spring 172.

The access opening 170 may include an abutment surface acting as an end stop such that when the plug 174 is fully inserted and tightened into the access opening 170, the plug 174 may abut the end stop. Tightening the plug 174 against the end stop may ensure that the spring 172 is compressed a desired amount for providing the desired force the inflow pump 60 exerts on the flexible pump tubing 106, as described further herein.

In some instances, the plug 174 may provide a mechanism to adjust the force the spring 172 applies to the pump mount 150. For example, tightening the plug 174 (e.g., rotating the plug 174 in a clockwise direction) may increase the compression of the spring 172 (i.e., further compress the spring 172) to increase the force the spring 172 exerts onto the pump mount 150 and/or loosening the plug 174 (e.g., rotating the plug 174 in a counter-clockwise direction) may reduce the compression of the spring 172 to decrease the force the spring 172 exerts onto the pump mount 150. It is noted that the compression force of the spring 172 is proportional to the force the inflow pump 60 exerts on the flexible pump tubing 106, as described further herein.

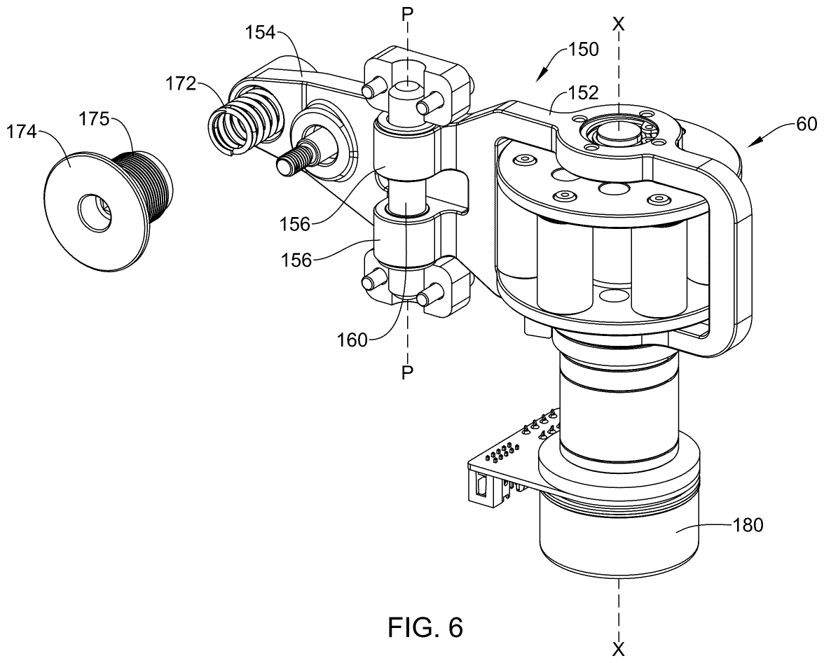

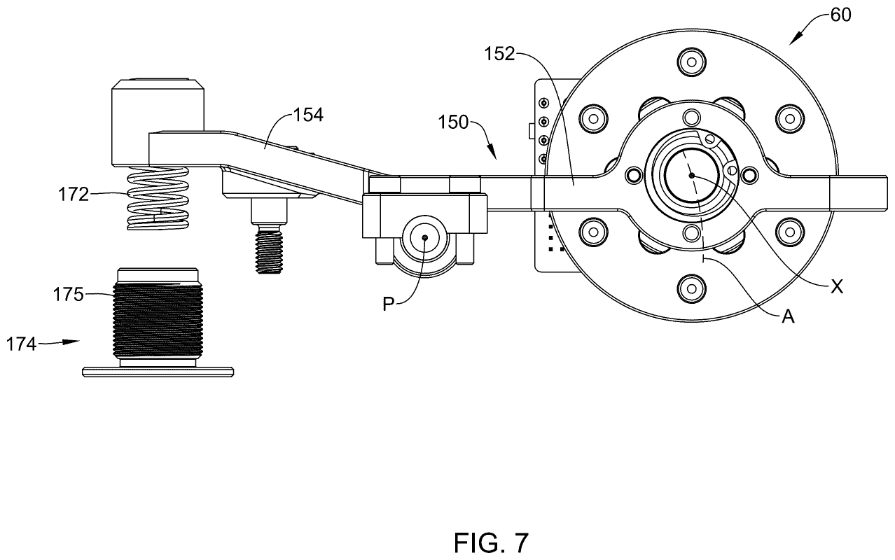

FIGS. 6 and 7 illustrate further aspects of the interaction of the spring 172 with the pump mount 150. As shown, the inflow pump 60 may be rotatably mounted to a pump mount 150 such that the inflow pump 60 rotates about a rotational axis X during operation. The pump mount 150 includes a pivot point P (e.g., fulcrum), a first arm 152 (e.g., load arm) extending from the pivot point P to the inflow pump 60, and a second arm 154 (e.g., effort arm) extending from the pivot point P away from the inflow pump 60. The pivot point P may also be referred to as a pivot axis about which the pump mount 150, and thus the inflow pump 60 pivots about. Thus, the inflow pump 60 may be configured to pivot relative to the housing 22 of the console 20 about the pivot axis P during operation of the inflow pump 60. As shown in FIG. 7, the pivot axis P may be spaced apart from the rotational axis X of the inflow pump 60. The pivot axis P may be parallel to the rotational axis X. The spring 172 may exert an input force onto the second arm 154, whereas the first arm 152 may output a force onto the inflow pump 60 via the rotatable connection between the inflow pump 60 and the first arm 152. Thus the compressive force of the spring 172 onto the second arm 154 may be proportional to the force the inflow pump 60 exerts on the flexible pump tubing 106.

In some instances, the pivotable connection between the pump mount 150 and the housing 22 of the console 20 may include a pivot pin 160 extending along the pivot axis P. For example, the pump mount 150 may include one or more bushings 156 configured to engage the pivot pin 160 to permit the pump mount 150 to pivot about the pivot pin 160, and thus the pivot axis P.

A motor 180 driving the inflow pump 60 is also shown in FIG. 6. The motor 180 may include a drive shaft aligned with the rotational axis X of the inflow pump 60. During operation, the motor 180 may move with the inflow pump 60 and the pump mount 150, and thus likewise pivot about the pivot pin 160, and thus the pivot axis P.

As shown in FIG. 7, the rotational axis X of the inflow pump 60 is configured to float (e.g., move) along an arcuate pathway A relative to the housing 22 of the console 20 during operation of the inflow pump 60. The arcuate pathway A may extend along a radius of the pivot axis P at a distance equal to the orthogonal distance between the pivot axis P and the rotational axis X. The motor 180 (e.g., the drive shaft of the motor 180) may also move along the arcuate pathway A with the inflow pump 60 during operation of the inflow pump 60.

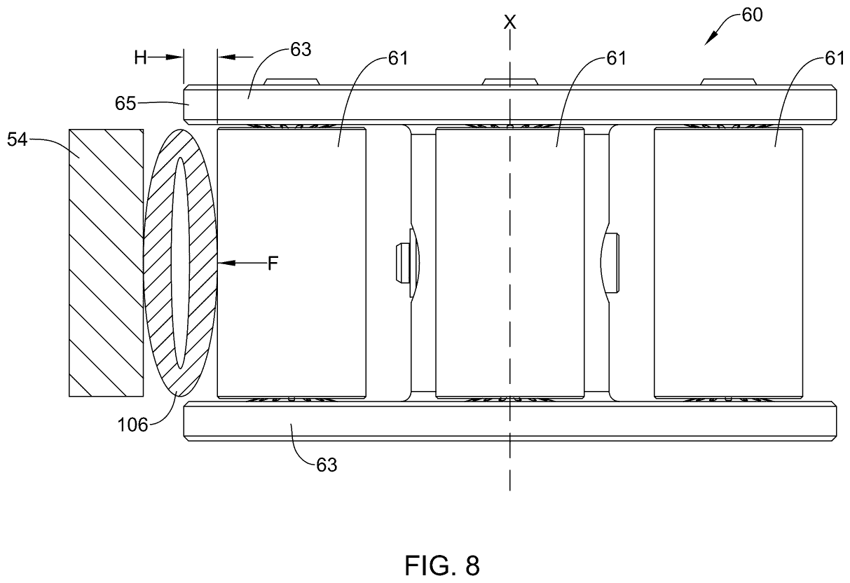

Referring to FIG. 8, the length of flexible pump tubing 106 of the cassette 110 may be compressed between the rollers 61 of the inflow pump 60 and the occlusion bed 54 with the inflow pump 60 exerting a compressive force F on the flexible pump tubing 106. For instance, the spring 172, as well as the length of the first and second arms 152, 154 of the pump mount 150 may be sized appropriately such that the inflow pump 60 is configured to apply a desired force against the length of flexible pump tubing 106 compressed between the inflow pump 60 and the occlusion bed 54. The described mounted inflow pump 60 may provide a constant or consistent force F on the flexible pump tubing 106. In some instances, the force F applied to the flexible pump tubing 106 by the inflow pump 60 may be in the range of 47.5 Newtons (N) to 74.5 N, in the range of 55 N to 65 N, in the range of 60 N to 65 N, about 60 N±5 N, about 62 N±2 N, or about 63 N±1 N, for example. Ensuring that the force F applied to the flexible pump tubing 106 by the inflow pump 60 is consistent helps maintain a consistent flow of fluid through the fluid cassette 110 and the outflow tubing 104. Ensuring a consistent force F may also mitigate premature degradation or damage of the flexible pump tubing 106.

Further aspects of the inflow pump 60 are shown in FIG. 8. The inflow pump 60 may be a roller pump including a plurality of rollers 61, each rotatably mounted between end plates 63 of the roller pump. The end plates 63 may extend radially outward of the rotational axis X of the inflow pump 60 beyond the outer extent of the rollers 61, providing an overhang 65. The overhang 65 of the end plates 63 may extend a radial distance H beyond the outer radial extent of the rollers 61. The overhangs 65 of the end plates 63 may help retain the flexible pump tubing 106 therebetween such that the flexible pump tubing 106 does not migrate toward one edge of the rollers 61 to mitigate premature degradation or damage of the flexible pump tubing 106.

FIG. 9 is a perspective view of a portion of the inflow pump 60 and associated mounting assembly (e.g., pump mount 150), including a fluid-tight containment housing 200. The fluid-tight containment housing 200 may be mounted on a rear face of the rear wall 56 of the receptacle 52 to provide a fluid-tight barrier between the portion of the console 20 housing the inflow pump 60 and the portion of the console 20 housing the controller, electrical circuitry and other components that must stay free of fluid that could damage the components. The fluid-tight containment housing 200 may include a seal 210 extending around a perimeter of the fluid-tight containment housing 200, such that the seal 210 is compressed against the rear face of the rear wall 56 of the receptacle 52 when the fluid-tight containment housing 200 is secured thereto. Thus, any fluid lost from the fluid tubing set 100 (e.g., the fluid cassette 110, or associated tubing 102, 104, 106) cannot reach the controller, electrical circuitry and other components within the console 20. One or more drainage tubes 220 may extend from a lower end of the fluid-tight containment housing 200 to drain any fluid collected in the fluid-tight containment housing 200. For example, captured fluid may be drained through the drainage tube(s) 220 onto the floor, in some instances. In some instance, the inflow pump 60 may be contained within the fluid-tight containment housing 200 while the motor 180 is positioned exterior of the fluid tight containment housing 200. In such an instance, the seal 210 may fluidly isolate the motor 180 from the inflow pump 60. For instance, the seal 210 may include a flexible boot 230 positioned between the motor 180 and the inflow pump 60. The flexible boot 230 may permit the floating movement of the inflow pump 60 (and the attached motor 180) relative to the housing 22 of the console 20, as described above, while providing a fluid barrier between the motor 180 and the inflow pump 60.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid management system comprising:
a console including a roller pump and a controller for operating the roller pump, the console including a housing and a door movable between a closed position and an open position;
wherein the roller pump is rotatable about a rotational axis during operation of the roller pump;
wherein the rotational axis is configured to float along an arcuate pathway relative to the housing during operation of the roller pump.

2. The fluid management system of claim 1, further comprising a fluid cassette insertable into a receptacle of the housing of the console;
wherein the fluid cassette includes a length of flexible pump tubing configured to engage the roller pump.

3. The fluid management system of claim 2, wherein the flexible pump tubing extends in an arcuate pathway in which the pump tubing extends outward beyond a front face of a housing of the fluid cassette when engaged with the roller pump.

4. The fluid management system of claim 2, wherein the door includes an occlusion bed mounted on an interior surface of the door, wherein the occlusion bed is configured to engage the length of flexible pump tubing of the fluid cassette when the door is closed to compress the length of flexible pump tubing between the occlusion bed and the roller pump.

5. The fluid management system of claim 2, wherein the roller pump is configured to apply a force against the length of flexible pump tubing in the range of 47.5 Newtons (N) to 74.5 N.

6. The fluid management system of claim 1, wherein the roller pump is rotatably mounted on a pump mount, wherein the pump mount is pivotably mounted to the housing.

7. The fluid management system of claim 6, wherein the pump mount includes a pivot point, a first arm extending from the pivot point to the roller pump, and a second arm extending from the pivot point away from the roller pump.

8. The fluid management system of claim 7, further comprising a spring exerting a force against the second arm of the pump mount.

9. The fluid management system of claim 8, wherein the spring is replaceable via a threaded plug of the housing of the console.

10. The fluid management system of claim 1, further comprising a motor driving the roller pump, wherein the motor moves along the arcuate pathway with the roller pump during operation of the roller pump.

11. The fluid management system of claim 1, further comprising a fluid-tight containment housing surrounding the roller pump, wherein the fluid-tight containment housing is configured to isolate the controller and associated electronic circuitry of the console from fluid.

12. A fluid management system comprising:
a console including a roller pump and a controller for operating the roller pump, the console including a housing and a door movable between a closed position and an open position;
wherein the roller pump is rotatable about a rotational axis during operation of the roller pump;
wherein the roller pump is configured to pivot relative to the housing about a pivot axis during operation of the roller pump, the pivot axis being spaced apart from the rotational axis.

13. The fluid management system of claim 12, wherein the pivot axis is parallel to the rotational axis.

14. The fluid management system of claim 12, further comprising a fluid cassette insertable into a receptacle of the housing of the console;
wherein the fluid cassette includes a length of flexible pump tubing configured to engage the roller pump.

15. The fluid management system of claim 14, wherein the flexible pump tubing extends in an arcuate pathway along roller pump.

16. The fluid management system of claim 14, wherein the door includes an occlusion bed mounted on an interior surface of the door, wherein the occlusion bed is configured to engage the length of flexible pump tubing of the fluid cassette when the door is closed to compress the length of flexible pump tubing between the occlusion bed and the roller pump.

17. The fluid management system of claim 14, wherein the roller pump is configured to apply a force against the length of flexible pump tubing in the range of 47.5 Newtons (N) to 74.5 N.

18. The fluid management system of claim 12, further comprising a fluid-tight containment housing surrounding the roller pump, wherein the fluid-tight containment housing is configured to isolate the controller and associated electronic circuitry of the console from fluid.

19. A method of operating a fluid management system, comprising:
positioning a housing of a fluid cassette of a disposable fluid tubing set into a receptacle of a housing of a console of the fluid management system with a length of flexible pump tubing of the fluid cassette engaged against a roller pump of the console; and
closing a door of the console such that an occlusion bed on an interior wall of the door compresses the length of flexible pump tubing between the occlusion bed and the roller pump;
wherein a rotational axis of the roller pump is configured to float along an arcuate pathway relative to the housing during operation of the roller pump.

20. The method of claim 19, wherein the roller pump is configured to apply a force against the length of flexible pump tubing in the range of 47.5 Newtons (N) to 74.5 N.
